(19) United States
(12) Patent Application Publication
(10) Pub. No.: US 2006/0035365 A1
(43) Pub. Date: Feb. 16, 2006

(54) Method for identifying cellular growth inhibitors

(76) Inventors: Michael J. Hamelin, Jersey City, NJ (US); Doris F. Cully, Scotch Plains, NJ (US)

Correspondence Address: MERCK AND CO., INC, P O BOX 2000, RAHWAY, NJ, 07065-0907, US

(21) Appl. No.: 10/521,300

(22) PCT Filed: Jul. 14, 2003
(86) PCT No.: PCT/US03/21726

§ 371 Date: Jan. 14, 2005

Related U.S. Application Data
(60) Provisional application No. 60/396,374, filed on Jul. 17, 2002.
(60) Provisional application No. 60/396,655, filed on Jul. 18, 2002.
(60) Provisional application No. 60/467,704, filed on May 2, 2003.

(51) Int. Cl. C12N 1/21; C12Q 1/18

(52) U.S. Cl. 435/252.3; 435/32

(57) ABSTRACT

The present invention is a method which permits the selective screening for growth-inhibiting substances having a known mechanism of action; i.e., substances which inhibit or otherwise interfere with an enzyme or other gene product whose function is required for the growth or survival of the cell. The method employs cells (e.g., bacterial cells) that contain a nucleic acid fragment that encodes an RNA fragment that can interfere with the expression of a gene product (e.g., an anti-sense RNA that hybridizes to a mRNA), wherein expression of the RNA fragment pre-sensitizes the cell to substances (drugs) that act at the gene product (e.g., a protein or RNA). The cells lose the capability to express the RNA fragment. In the method, the recombinant cells are grown in a nutrient medium in the presence of a test substance under conditions in which expression of the RNA fragment occurs at a level that pre-sensitizes the cell to substances that act at the targeted gene product. The growth conditions are also controlled such that the cells lose the capability to express the RNA fragment. When the test substance is a growth inhibitor that acts on the targeted gene product, the cells lacking the RNA fragment (revertant cells) will have a growth advantage over cells containing the RNA fragment, and the growth of revertant cells will occur. The method of the invention includes monitoring the cell growth for the appearance of revertant cells, which leads to the identification of selective growth inhibitors having a specific mode of action.

### FIELD OF INVENTION

The invention is directed to a method for identifying substances that inhibit the growth of cells by acting on a particular gene product required for cell growth. In one embodiment, the method is employed to identify substances that are antibacterial agents having a particular mechanism of action, such as the inhibition of an enzyme necessary for fatty acid biosynthesis. The method of the invention utilizes cells which encode an RNA fragment (e.g., antisense RNA) that can interfere with the expression of the gene encoding the target gene product, such that the expression of the RNA fragment pre-sensitizes the cell to substances that act at the gene product. The method also utilizes conditions under which the cells lose the capability to express the RNA fragment, leading to cells that can grow in the presence of a substance that acts at the target gene product (referred to herein as “revertant” cells). The detection via the method of the invention of revertant cell colonies in the presence of a given substance identifies that substance as a growth inhibitor that acts at the target gene product.

### BACKGROUND OF THE INVENTION

Pathogenic strains of bacteria which represent a major threat to public health include Staphylococcus, Streptococcus, Enterococcus, Escherichia, Klebsiella, Haemophilus, Enterobacter, Acinetobacter, Bacillus, Stenotrophomonas, Salmonella, Burkholderia, and Pseudomonas, specifically including the strains Streptococcus pneumonia, Staphylococcus aureus, Enterococcus faecalis, Enterococcus faecium, Escherichia coli, Klebsiella pneumoniae, Haemophilus influenzae, Enterobacter cloacae, Pseudomonas aeruginosa, Acinetobacter baumanii, Bacillus subtilis, Stenotrophomonas maltophilia, Salmonella typhimurium, and Burkholderia cepacia. Pathogenic bacteria cause such diseases as pneumonia, typhoid, diarrhea, and tuberculosis. Antibiotic agents have been developed to combat such diseases, including, for example, amikacin, gentamicin, tobramycin, amoxicillin, amphotericin B, ampicillin, atovaquone, azithromycin, cefazolin, cefepime, cefotaxime, cefotetan, cefpodoxime, ceftazidime, ceftizoxime, ceftriaxone, cefuroxime, cephalexin, chloramphenicol, clotrimazole, ciprofloxacin, clarithromycin, clindamycin, dicloxacillin, doxycycline, erthromycin lactobionate, imipenem, izoniazid, kanamycin, linezolid, metronidazole, nafcillin, nitrofurantoin, nystatin, penicillin, pentamidine, piperacillin, rifampin, ticarcillin, trimethoprim, and vancomycin.

While these agents are effective against pathogenic bacteria and are thus useful in the treatment of disease conditions associated with the presence of bacteria, many of the clinically important strains of bacteria are becoming resistant to the known antibiotics. For example, Enterococci that are resistant to a vast array of antimicrobial drugs, including cell wall active agents, aminoglycosides, penicillin, ampicillin, and vancomycin, have been observed. Staphylococcus aureus, the most frequent causative agent of infections in the hospital environment, is resistant to many of the known antibiotics, making it difficult to treat such infections. Further description of the nature of antibiotics and the development of antibiotic resistance is found in Walsh, Nature 2000, 406: 775-781. Accordingly, there is a continuing need to discover new antibacterial agents with novel mechanisms of action, and thus a low probability for preselected resistance in the clinic, to combat the rise of resistant strains in both the hospital and community environments. There is a concomitant need to develop reliable, high-throughput methods for identifying new antibacterials having a specific mechanism of action and bacterial cell wall permeability.

Pathogenic strains of fungi which represent a major threat to public health include: Cryptococcus spp., Candida spp., Aspergillus spp., Histoplasma spp., Coccidioides spp., Paracoccidioides spp., Blastomyces spp., Fusarium spp., Sporothrix spp., Trichosporon spp., Scedosporium, Rhizopus spp., Pseudallescheria spp., dermatophytes, Paeciliomyces spp., Alternaria spp., Curvularia spp., Exophiala spp., Schizosaccharomyces spp., Wangiella spp., Dematiaceous fungi and Pneumocystis spp., and specifically Saccharomyces cerevisiae, Aspergillus nidulans, Aspergillus fumigatus, Cryptococcus neoformans, Coccidioides immitis, Schizosaccharomyces pombe, Pneumocystis carinii, and Candida albicans. Current therapies to these fungi are inadequate due to a poor spectrum of activity and to toxicity. Resistance to antifungals is not as prevalent as found with antibacterial agents, however there is a steady increase in resistance to antifungal agents used in a hospital setting. The immuno-compromised patient is particularly susceptible to fungal infections where drug interactions with existing antifungal therapies can be an issue. There is a need for new antifungals with novel mechanisms of action that exhibit improved therapeutic profiles.

The standard approach for the discovery of antibacterials is to expose a test compound or mixture of compounds to the bacterial cells, and measure the level of cellular growth inhibition. Growth inhibition or cell death is typically determined in a liquid medium by measuring optical density of the culture following an 18-hour growth period. The growth inhibition of a test compound is typically reported in terms of minimum inhibitory concentration (MIC) required to inhibit 99.9 percent of the cell growth or where no visible growth is observed. Such assays are described, for example, in W. Hewitt, Microbiology Assay (Academic Press, New York, N.Y., 1977), pp. 1-261. When liquid culture-based assays are performed on a single concentration of compound, they are economical, amenable to high-throughput screening (HTS) and suited for the screening of compounds soluble in low levels of solvent (e.g., 0.2% DMSO). The disadvantages of HTS liquid cell growth assays are the limited capability to measure water insoluble compounds, the difficulty in determining a compound's relative potency, the repeated identification of commonly known antibacterials (i.e., high rate of redundancy), and a predisposition to identify compounds which are potent but also toxic to mammalian cells.

A semi-solid medium approach for discovering bacterial growth inhibitors is the agar zone of inhibition assay, wherein agar in a dish or plate is impregnated with bacteria and the compound is applied to the plate either on a paper disk, in a precast well, or as a drop on the agar surface. Bacterial growth inhibition or cell death is typically determined by measuring zones of inhibition in the assay plate. The conventional agar assay (or, more generally, the semi-solid medium-based assay) is further described, for example, in J. F. Acar and F. W. Goldstein, “Procedure for testing antimicrobial agents in agar media: theoretical considerations” in: Antibiotics in Laboratory Medicine, edited by V. Lorian (Williams & Wilkins, Baltimore Md., 1996), pp 1-51. Semi-solid medium-based assays are well-suited for solid samples and fresh natural products and extracts of natural products Difficulties in the rapid and accurate detection and measurement of inhibition zones restrict the use of semi-solid-medium based assays for high-throughput screening. Another difficulty with these screens is the high rate of redundancy in the mode of action of identified active agents; e.g., compounds acting at cell wall components are found with a high frequency.

Biochemical, in vitro-based assays can also be used for antibacterial discovery. An advantage of this approach is that it permits the rapid identification of substances acting at a very specific (and pre-determined) target. A major disadvantage of this mechanism-based approach has been the identification of compounds that exhibit potent biochemical inhibitory activity but lack the cell wall permeability needed for an effective antibacterial.

A resistant bacteria can be used in a screen for selective target screening. Drug resistant and sensitive strains can be tested in liquid or semi-solid-media and the differential sensitivities measured. This approach uses a mutated target gene product to select for an antibacterial that can penetrate the cell wall. For example, to screen for antibiotics that act like ampicillin, an ampicillin-sensitive strain can be compared to a resistant strain wherein any compound affecting the sensitive strain but not the resistant one is singled out as positive. The screening for antibacterials on a drug resistant mutant bacteria will allow the identification of antibacterials that act at that genetic site, but will frequently miss the antibacterials that act at other regions of the target. An example is an antibiotic that acts by inhibiting protein synthesis by binding to ribosomes, where cross-resistance with ribosomal binding drugs is frequently not observed. Differential resistant/sensitive assays do not screen on the target gene product, but on selective mutant products. Resistant/sensitive pair screens performed in liquid and agar assays suffer from the same disadvantages outlined above.

An example of target selective screening for antibacterials in whole cells is outlined in Forsyth et al., Mol. Microbiol. 2002, 43: 1387-1400, which discloses that the expression of antisense RNA (hereinafter asRNA) to genes required for Staphylococcus aureus growth results in an increased sensitivity to targeted antibacterials. The asRNA assay outlined in Forsyth et al. is performed in a liquid-medium and requires the comparison with a non-sensitized strain, with a 7-point titration performed in duplicate. The disadvantages of the liquid-medium asRNA assay are that it requires the comparison of two strains for every sample tested, it is not amenable to high-throughput screening (due to titrations and variability), compounds of low water solubility will be difficult to measure in a full dose titration, a large amount of sample compound is required, and extracts or purified compounds are required.

Other examples of sensitization by altering the expression of bacterial genes include Ji et al., Science 2001, 293: 2266-2269, directed to Identification of critical staphylococcal genes using conditional phenotypes generated by antisense RNA; and DeVito et al. Nat. Biotechnol. 2002, 20: 478-483, directed to target-specific screening strains for antibacterial discovery.

While most of the discussion in this section has focused on known methods for discovering antibacterials, the same or similar methods have been employed for the discovery of antifungals, methods that suffer from the same or similar disadvantages.

There is a need for the development of new and/or improved antimicrobial screening methods that minimize or avoid the drawbacks associated with known assays. In particular, there is a need for mechanism-based, whole cell screening methods amenable to high throughput.

### SUMMARY OF THE INVENTION

The present invention is a method which permits the selective screening for growth-inhibiting substances (e.g., compounds or natural products) having a known mechanism of action; i.e., substances which inhibit or otherwise interfere with enzymes or other gene products of the target cell whose functions are required for the growth or survival of the cell. As used herein, the term “inhibiting” (or “inhibition”) in reference to cellular growth means the reduction or suppression of growth of the cells (e.g., bacterial cells). The method of the invention utilizes recombinant cells containing nucleic acid that encodes an RNA fragment (e.g., antisense RNA) whose expression can decrease the amount of a targeted gene product in the cell. Antisense RNA (asRNA), for example, can hybridize to the messenger RNA (mRNA) encoding a targeted gene product (e.g., a protein), and thereby interfere with the expression of the gene product. Depending upon the level of expression of the RNA-encoding nucleic acid induced in the cell, the production of the targeted gene product can either be reduced (i.e., down-regulated) drastically leading to growth arrest of the cell or can be reduced to a level that leaves cellular growth essentially unaffected but increases the sensitivity of the cell to substances that act on the gene product. For example, if the targeted gene product is an enzyme required for cellular growth, expression of the asRNA can pre-sensitize the cell to substances that inhibit the enzyme. Under certain controllable conditions, the cells can become unstable in the sense that they can lose the asRNA-encoding nucleic acid, or the ability to express the asRNA. Cells which have lost the asRNA-encoding nucleic acid or the ability to express the asRNA are referred to herein as revertant cells. Revertant cells are able to grow in the presence of a substance that acts on the targeted gene product.

In the method of the present invention, the recombinant cells are grown in a nutrient medium in the presence of a test substance under conditions in which expression of the RNA fragment occurs at a level that pre-sensitizes the cell to substances that act at the targeted gene product. The growth conditions are also controlled such that a fraction of the cells lose the capability to express the RNA fragment. If the test substance is a growth inhibitor that does not act at the targeted gene product, it will be equally potent on cells containing the RNA fragment and on cells lacking the RNA fragment (revertant cells). The revertant cells will not have a growth advantage and essentially all of the cells will be killed; i.e., no growth will occur. On the other hand, if the test substance acts on the targeted gene product, the cells lacking the RNA fragment will have a growth advantage over cells containing the RNA fragment, and the growth of revertant cells will occur. When the nutrient medium used to grow the cells is a semi-solid nutrient medium, because the density of revertant cells is intrinsically very low (compared to the non-revertant cells), their preferential growth in the selective area of the inhibition zone can be seen as isolated, individual colonies. These revertant colonies are also large enough to be detected by visual inspection. If the test substance is not a growth inhibitor, there will be no inhibition of growth. Analyzing the cell growth for the appearance of revertant cells leads to the identification of growth inhibitors having a specific mode of action.

More particularly, the present invention is a method for testing whether a substance is or contains a cell growth inhibitor that acts by selectively inhibiting the function of a gene product required for cellular growth or survival, wherein the method comprises:

(A) providing recombinant cells that are capable of expressing an RNA fragment that interferes with the expression of the gene product;

(B) growing the recombinant cells in a nutrient medium in the presence of the test substance and under conditions which result in (i) the expression of the RNA fragment and down regulation of the synthesis of the gene product and (ii) the loss by the cells of the capability to express the RNA fragment; and

(C) analyzing the resulting cell growth; wherein:
(1) if there is essentially no cell growth due to the death of all or substantially all of the cells, then the test substance is or contains a growth inhibitor that does not selectively inhibit the targeted gene product;
(2) if there is essentially no inhibition of cell growth due to the survival and growth of all or substantially all of the cells, then the test substance is not or does not contain a growth inhibitor; or
(3) if there is growth inhibition due to the death of a substantial portion of the cells accompanied by the survival and growth of revertant cells having no capability to express the RNA fragment, then the test substance is or contains a growth inhibitor that selectively inhibits the targeted gene product.

An embodiment of the present invention is the method as just described, wherein the RNA fragment expressed by the cells in Step A comprises an antisense RNA. In an aspect of this embodiment, the antisense RNA is encoded in a plasmid contained in the cells. In another aspect of this embodiment, the antisense RNA is encoded in DNA in the genome of the cells.

Another embodiment of the present invention is the method as originally set forth above, wherein the cells employed in the method are selected from the group consisting of bacterial strains and fungal strains. In an aspect of this embodiment, the cells are a strain of bacteria and the substance is being tested to determine whether it is or contains an antibacterial agent that acts by selectively inhibiting the function of a gene product required for the growth or survival of the bacteria. In a feature of the preceding aspect, the RNA fragment capable of being expressed by the bacteria in Step A comprises an antisense RNA encoded in a plasmid contained in the bacteria.

In a preferred embodiment of the method of the present invention, the nutrient medium in which the recombinant cells (e.g., bacterial cells) are grown (Step B) is a semi-solid medium (which may alternatively be referred to herein as a semi-solid growth medium or a semi-solid nutrient medium), such as an agar plate, an agarose plate, or any solid-support matrix wherein the semi-solid medium is inoculated with the test substance and the pattern of cell growth on the plate is analyzed (Step C). The presence at the inoculation site of a zone of growth inhibition having one or more visually detectable revertant cell colonies in and/or around the zone indicates that the test substance acts at the targeted gene product and has a specific mode of action. On the other hand, the presence at the inoculation site of a clear zone of no cellular growth (i.e., no detectable revertant cell colonies) indicates that the test substance is or contains a growth inhibitor that does not act at the targeted gene product. In a preferred aspect of this embodiment, the cells are bacterial cells.

The method of the present invention overcomes many of the drawbacks of the known assay methods as described in the Background of the Invention. More particularly, the present invention is a mechanism-based whole cell screen; it is performed with a single sample point; it does not require dose titrations of sample; it is amenable to high throughput screening; it can measure water insoluble compounds; it eliminates the identification of generally toxic compounds; it has a rapid assay readout; it assays cell permeability; it performs direct screening on intact gene product; it can be performed on a single assay plate with a single bacterial or antifungal strain; and it can measure crude compounds produced from microbial sources (extracts and living colonies).

Various other embodiments, aspects and features of the present invention are either further described in or will be apparent from the ensuing description, examples and appended claims.

### BRIEF DESCRIPTION OF THE DRAWINGS

FIG. 1 shows the growth inhibition zone for the test substance thiotetramycin on the assay plate resulting from the assay set forth in Example 1 for identifying fabF-acting substances in S. aureus. The zone has several visually observable revertant bacterial colonies.

FIG. 2 shows the growth inhibition zone for the cerulenin control on the assay plate resulting from the assay set forth in Example 1 for identifying fabF-acting substances in S. aureus. Similar to the test substance in FIG. 1, the zone has several visually observable revertant bacterial colonies.

FIG. 3 shows the growth inhibition zone for the kanamycin control on the assay plate resulting from the assay set forth in Example 1 for identifying fabF-acting substances in S. aureus. The zone has no visually observable bacterial growth.

FIG. 4 shows sections of assay plates resulting from the strip assay set forth in Example 2 for identifying fabF-acting substances in S. aureus from unidentified microorganisms isolated from soil samples.

FIG. 5 shows the growth inhibition zone for an undefined extract containing antimicrobial-producing soil microorganisms resulting from the assay set forth in Example 2 for identifying fabF-acting substances in S. aureus. The zone has several visually observable revertant bacterial colonies surrounding a central zone of no visually observable bacterial growth.

FIG. 6 presents plots of the Relative Fluorescence Index for a xylose-inducible S. aureus SafabF/GFP strain in response to treatment with various concentrations of fabF- and non-fabF-acting substances in the liquid-based assay set forth in Example 3.

### DETAILED DESCRIPTION OF THE INVENTION

The present invention is a method for testing whether a substance is or contains a cell growth inhibitor that acts by selectively inhibiting the function of a gene product required for cellular growth or survival. The method employs recombinant cells (e.g., bacterial cells or fungal cells) that are capable of expressing an RNA fragment that interferes with the expression of the gene product. The target gene is a gene that is required for the growth or survival of the host cell. The RNA fragment can be an asRNA that, when expressed, hybridizes to and interferes with the expression of the target gene leading to down regulation of the targeted gene product. This down regulation in turn results in increased sensitivity of the antisense-expressing cells (e.g., bacteria) to growth inhibitors (e.g., antibiotics) acting at the targeted gene product. At the same time, the cells are unstable with respect to RNA fragment expression in that the cells can lose the capability to express the RNA fragment under certain growth conditions. As noted above, in the method of the present invention, these cells are grown in the presence of a test substance under conditions which concurrently result in expression of the RNA fragment and the loss over time of this expression capability. The term “loss” in this context means a gradual loss by the cells of the capability to express the RNA fragment; i.e., the growth and replication of cells in Step B is conducted such that over time a portion of the cells lose the capability to express the RNA fragment, these cells becoming the source for revertant colonies. The cell growth is monitored under these conditions for the detection/appearance of revertant cells which will have a growth advantage when the test substance acts at the targeted gene product.

The recombinant cells can be induced to express the RNA fragment by contact with a suitable inducing agent. In addition the cells can contain an antibiotic resistance gene which stabilizes (i.e., sustains) the cells' capability to express the RNA fragment by continuous selection with the antibiotic. In this case, growth under conditions which result in (i) the expression of the RNA fragment (e.g., asRNA) and down regulation of the synthesis of the gene product and (ii) the loss by the cells of the capability to express the RNA fragment means growing the cells in the presence of an effective amount of the RNA fragment-expression inducer and in the absence of the antibiotic. An effective amount of the inducer means an amount that sensitizes the cells to a growth inhibitor acting on the targeted gene product. Too low an amount of inducer will result in insufficient RNA induction and a corresponding lack of cell sensitivity to selective inhibitors, in which case revertant cells will have no selective growth advantage. Too high an amount of inducer will lead to overexpression of the asRNA, which will arrest cellular growth altogether irrespective of the nature of the test substance. An effective amount of inducer for use in the present invention can be established through the use of controls. For example, as a check that an effective amount of inducer is being employed in the method of the invention, the cells can be grown concurrently under conditions identical to those used for the test substance(s), wherein the test substance is replaced with a growth inhibitor known to act selectively at the targeted gene product. The cells can optionally also be grown with a growth inhibitor that is known not to act selectively at the targeted gene product. If the inducer is present in an effective amount, the run with the known selective growth inhibitor will result in the growth of revertant cells, whereas the run with the known non-selective inhibitor will result in no growth. If the controls provide contrary or inconclusive results, then the run(s) with the test substance(s) can be considered not valid.

The recombinant cells can be grown using conventional microbiological media and culture conditions (e.g., LB-agar), and such conditions can be found in W. Hewitt, Microbiology Assay, (Academic Press, New York, N.Y., 1977).

The present invention can be conducted in a liquid medium or in a semi-solid medium. Revertant cells can be detected in a liquid medium by taking advantage of the instability of the RNA fragment. For example, when an asRNA-encoding plasmid is employed, the plasmid can be constructed so that a repressor protein is deleted with the asRNA-encoding nucleic acid. The cell can also have a reporter gene that is under the regulation of the repressor. Accordingly, when the cells are grown under conditions that render the asRNA-encoding nucleic acid unstable (i.e., under conditions in which the cells lose the asRNA-containing plasmid and/or develop a non-functional partial plasmid), the deletion of the repressor will result in the activation of the reporter gene. The reporter gene can be a green fluorescent protein and can be integrated into the cellular genome or carried on a separate plasmid (distinct from asRNA-encoding nucleic acid) in the cell. The resulting revertant colonies will be fluorescent and detectable in a liquid medium (and in a semi-solid medium as well). This reporter system can be used when the asRNA-encoding nucleic acid is present in the bacterial genome, and not in a separate plasmid.

The method of the present invention is preferably conducted in a semi-solid medium (i.e., a semi-solid nutrient medium). Accordingly, an embodiment of the present invention is a method for testing whether a substance is or contains a cell growth inhibitor that acts by selectively inhibiting the function of a gene product required for cellular growth or survival, wherein the method comprises:

(A) providing recombinant cells that are capable of expressing an RNA fragment that interferes with the expression of the gene product;

(B) growing the recombinant cells in a semi-solid medium in the presence of the test substance (e.g., employing a medium inoculated with the test substance) and under conditions which result in (i) the expression of the RNA fragment and down regulation of the synthesis of the gene product and (ii) the loss by the cells of the capability to express the RNA fragment; and

(C) analyzing the resulting cell growth; wherein:
(1) if the semi-solid medium exhibits a clear zone (e.g., a clear zone in and around the test substance inoculation site) indicative of essentially no growth due to the death of all or substantially all of the cells, then the test substance is or contains an antibacterial agent that does not selectively inhibit the targeted gene product;
(2) if the semi-solid medium does not exhibit a zone of no growth, due to the survival and growth of all or substantially all of the cells, then the test substance is not or does not contain an antibacterial agent; or
(3) if the semi-solid medium exhibits a zone of no growth except for one or more small cell colonies in the zone due to the survival and growth of revertant cells having no capability to express the RNA fragment, then the test substance is or contains an antibacterial agent that selectively inhibits the targeted gene product.

In an aspect of this embodiment, the semi-solid medium is an agar plate or an agarose plate. In another aspect of this embodiment, the RNA fragment expressed by the cells in Step A comprises an antisense RNA. In a feature of this aspect, the antisense RNA is encoded in DNA in the genome of the cells. In another feature of this aspect, the antisense RNA is encoded in a plasmid contained in the cells. The plasmid can be induced to express the asRNA by contact with a suitable inducer such as a sugar (e.g., xylose or arabinose), or anhydrotetracyline, or isopropyl-β-D-thiogalactopyranoside (IPTG). The plasmid can further contain an antibiotic resistance gene, which permits the plasmids to be sustained in the cells by continuous selection with the antibiotic to which the plasmid is resistant. In this case, growth under conditions which result in (i) the expression of the asRNA and down regulation of the synthesis of the gene product and (ii) the loss by the cells of the capability to express the RNA fragment means growing the cells in the presence of an effective amount of the asRNA-expression inducer and in the absence of the antibiotic. The loss of this capability can be due to the loss of the plasmid from the cells (i.e., the cells are “cured”) and/or to gene rearrangement within the plasmid. Plasmid instability and susceptibility to rearrangements are discussed in N. C. Franklin, “Illegitimate recombination”, in The Bacterio-phage Lambda, edited by A. D. Hershey, (Cold Spring Harbor Laboratory, New York, 1971) pp. 175-194, and in Ehrlich et al., Gene 1993, 135: 161-166. As noted earlier, an effective amount of the inducer means an amount that sensitizes the cells to a growth inhibitor acting on the targeted gene product. In this context, too low an amount of inducer will result in insufficient asRNA induction and a corresponding lack of cell sensitivity to selective inhibitors, in which case revertant cells will have no selective growth advantage. Too high an amount of inducer will lead to overexpression of the asRNA, which will arrest cellular growth altogether irrespective of the nature of the test substance. The validity of the results obtained for a test substance can be checked through the use of controls in the manner already described above.

Further embodiments of the present invention include the method conducted in a semi-solid medium as first described just above, incorporating two or more of the foregoing aspects or features. The nucleic acid fragment encoding an antisense RNA can also be incorporated into the genome of the cell using methods known to those skilled in the art. The loss of the capability to detect selective inhibitors (as described above) can be due to the loss of the asRNA-encoding nucleic acid by deletion or rearrangement of the genomic DNA. The genomic copy of the asRNA-encoding nucleic acid will be under the same induction mechanisms outlined above, where an inducer, such as xylose, arabinose, anhydrotetracycline or IPTG will enable the synthesis of asRNA. Exposure to compounds that act at the asRNA gene target will result in the selective deletion of the asRNA-encoding nucleic acid from the genome and revertant colonies will grow. For genomic instability, the asRNA-encoding nucleic acid would be integrated into the genome in a DNA fragment with a high recombination frequency (see, e.g., Hashem, V I et al. Mutat Res. 2002, 22: 39-46).

The method of the present invention is best practiced using a semi-solid medium, which has advantages over liquid medium assays. The semi-solid medium assay can be performed over a large range of sample concentrations or on crude non-quantifiable samples, which minimizes sample handling before performing the screen and avoids dose titrations, time consuming extract preparation, and pre-growth of large quantities of compound producing organisms. Unlike the liquid medium assay, the semi-solid medium assay can accommodate high concentrations of organic solvent, allowing for the assay of water insoluble compounds. The semi-solid medium assay format is amenable to screening of solid fresh samples such as bacterial colonies, fungal colonies, or even crude soil samples, or with samples that cannot be readily replicated. In the context of the current invention, the semi-solid medium assay also alleviates the need for inserting a reporter gene in a plasmid or the host cell genome. Rapid visual detection of growing revertant colonies within zones of inhibition suffices to identify positives.

An exemplary procedure for conducting the method of the present invention via a semi-solid medium is as follows: A suitable dose of the recombinant cells suspended in a broth (e.g., LB medium as described in Molecular Cloning: A Laboratory Manual, 3^{rd} edition, edited by J. Sambrook and D. W. Russell (Cold Spring Harbor Laboratory Press, 2000)) or solvent is added to semi-solid medium in a molten state (e.g., molten agar) supplemented with an agent for inducing expression of the. RNA fragment and optionally with other nutrients (e.g., glucose). The dosage of cells employed is typically in an amount that provides a range of from 10⁶ to 10⁸ cells per mL of semi-solid medium (e.g. agar). (The cells can be “counted” by suspending them in a liquid culture, measuring the optical density of the suspension—e.g., at 600 nm—, separating the cells from the medium, and then re-suspending the cells in the appropriate amount of the broth or solvent added to the semi-solid medium.) After mixing, the molten mixture is poured into dishes, after which wells can optionally be cast into the medium using a casting template. After the dishes cool to room temperature, samples of the substances to be tested plus sample of controls, each dissolved in a suitable solvent (e.g., DMSO) (the concentration of the substance in the solution is typically in a range of from about 0.001 to about 5 mg per mL), are dispensed (e.g., in an amount in a range of from about 1 to about 200 μL of the solution) into the wells and the plates incubated at a suitable temperature (e.g., from about 25 to about 45° C.) for a sufficient time (e.g., from about 12 to about 48 hours) to permit growth of the cells, and also under conditions in which the asRNA-encoding nucleic acid is not sustained in the cells (e.g., in the absence of an antibiotic for which the cells contain an antibiotic resistance gene). The incubated plates are then visually inspected for the appearance of revertant colonies in a zone around each test well in order to determine whether the test substances are or contain growth inhibitors acting at the targeted gene product.

The procedure just described can be modified to test for natural products that act as growth inhibitors with a specific mode of action. The assay plate can be prepared as described in the preceding paragraph, except that wells are not cast into the plate. Instead of adding the liquid test sample into a well on an assay plate, a strip of agar onto which microorganisms have been grown (e.g., from soil samples) is applied to the surface of the agar assay plate. Natural products produced by the microorganisms diffuse out of the strips and into the agar test plate. The incubated plates are then visually inspected for the appearance of revertant colonies in a zone around each microorganism-containing test strip, in order to determine whether the test substances are or contain growth inhibitors acting at the targeted gene product.

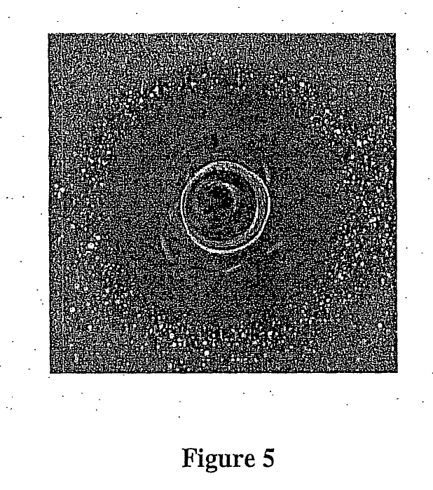

The terms “test substance” and “substance” are used interchangeably and refer to a compound, a mixture of compounds (i.e., at least two compounds), or a natural product sample containing one or more compounds. Thus, the analysis of the cell growth of a test substance may encompass the inhibitory activity of more than one growth inhibitor, such that combinations of selective and non-selective inhibitors of the targeted gene product will be visually observable. The method of the present invention is generally employed to identify selective growth inhibitors. When a test substance is a single compound and is identified as a selective growth inhibitor in the method of the present invention (i.e., the analysis of the cell growth in Step C of the method of invention determines that the growth falls into category (3)—the survival and growth of revertant cells), then the test substance “is” a selective growth inhibitor. When a test substance is a mixture of compounds (e.g., a natural product sample) and is identified as a selective growth inhibitor in the method of the present invention (i.e., the analysis of the cell growth in Step C of the method of invention determines that the growth falls into category (3)), then the test substance “contains” a selective growth inhibitor. More particularly, when the test substance is a mixture of compounds comprising a selective and a non-selective growth inhibitor, the analysis for cell growth in the method of the present invention (Step C) will show substantially no cell growth due to the non-selective growth inhibitor and will concurrently show the survival and growth of revertant cells having no capability to express the RNA fragment due to the selective inhibition of the targeted gene product by the selective growth inhibitor. For example, when the test substance is a mixture of compounds comprising a selective and a non-selective growth inhibitor and the method of the present invention is conducted using a semi-solid nutrient medium as heretofore described, the analysis for cell growth (Step C) will show one or more zones of no growth due to the non-selective growth inhibitor and in addition will show one or more zones of no growth except for one or more small cell colonies due to the survival and growth of the revertant cells. This type of growth—which is exemplified in FIG. 5—falls into category (3) under Step C, and the test substance “contains” a selective growth inhibitor.

The term “substantially all” with respect to the death of the cells employed in the method of the present invention means that little or no cell growth can be detected. In the context of growth on a semi-solid medium such as an agar plate, “substantially all” means that no cellular growth is visually observable; e.g., the putative growth zone is clear of growth to the naked eye as compared to the non-treated area. In the context of growth in a liquid medium, “substantially all” means that there is at least about 90%, typically at least about 95% (e.g., at least about 99%), and preferably about 99.9% growth inhibition of the cells, as determined for example by the change in the optical density of the medium.

The term “essentially no cell growth” with respect to the death of the cells employed in the method of the present invention means that little or no cell growth can be detected. In the context of growth on a semi-solid medium such as an agar plate, “essentially no cell growth” means that no cellular growth is visually observable; e.g., the putative growth zone is clear of growth to the naked eye as compared to the non-treated area. In the context of growth in a liquid medium, “essentially no cell growth” means that there is at least about 90%, typically at least about 95% (e.g., at least about 99%), and preferably about 99.9% growth inhibition of the cells, as determined for example by the change in the optical density of the medium.

Bacteria suitable for use in the present invention can be any recombinant bacteria known in the art that contains a genetic element encoding an RNA fragment whose expression interferes with the expression of a gene encoding a gene product required for cell growth or survival, and thereby renders the bacteria sensitive to the class of drugs (antibiotics) acting on that gene product. The bacteria can be pathogenic bacteria. Exemplary of the bacteria suitable for use in the present invention is a bacterial strain selected from the group consisting of Staphylococcus, Streptococcus, Enterococcus, Escherichia, Klebsiella, Haemophilus, Enterobacter, Acinetobacter, Bacillus, Stenotrophomonas, Burkholderia, Salmonella, and Pseudomonas, specifically including the strains Streptococcus pneumonia, Staphylococcus aureus, Enterococcus faecalis, Enterococcus faecium, Escherichia coli, Klebsiella pneumoniae, Haemophilus influenzae, Enterobacter cloacae, Pseudomonas aeruginosa, Acinetobacter baumanii, Stenotrophomonas maltophilia, Salmonella typhimurium, and Burkholderia cepacia. A particular example of bacteria suitable for use in the present invention is a bacterial strain is selected from the group consisting of Staphylococcus aureus, Streptococcus pneumoniae, Bacillus subtilis, Enterococcus faecalis, Escherichia coli, and Pseudomonas aeruginosa. A more particular example is a strain of Staphylococcus aureus, such as a Staphylococcus aureus containing a plasmid expressing a xylose-inducible anti-sense RNA for a gene required for the survival or growth of Staphylococcus aureus. Genes required for the survival or growth of S. Aureus include those selected from the group consisting of fabF, pheT, murA, secA, dnaC, ileS, ftsZ, secI, polC, dnaE, gyrE, gyrA, murB, rpl, parE, and parC. A sub-class of the genes required for the survival or growth of S. aureus includes those encoded to express a fatty acid synthase, an aminoacyl-tRNA synthetase, a protein secretase, a peptidyl transferase, a transglycosylase, a transpeptidase, or a ribosomal associated protein.

Methods for the growth and preservation of bacterial strains are disclosed in Molecular Cloning: A Laboratory Manual, 3^{rd} edition, edited by J. Sambrook and D. W. Russell (Cold Spring Harbor Laboratory Press, 2000). Strains of bacteria are available from the ATCC (American Tissue Culture Collection, Atlanta, Ga.)

The fungal strain employed in the method of the present invention can be any recombinant fungi known in the art that contains a genetic element encoding an RNA fragment whose expression interferes with the expression of a gene encoding a gene product required for cell growth or survival, and thereby renders the bacteria sensitive to the class of drugs (antifungals) acting on that gene product. Suitable fungal strains that can be used include: Cryptococcus spp., Candida spp., Aspergillus spp., Histoplasma spp., Coccidioides spp., Paracoccidioides spp., Blastomyces spp., Fusarium spp., Schizosaccharomyces spp., Sporothrix spp., Trichosporon spp., Scedosporium, Rhizopus spp., Pseudallescheria spp., dermatophytes, Paeciliomyces spp., Alternaria spp., Curvularia spp., Exophiala spp., Wangiella spp., Dematiaceous fungi and Pneumocystis spp., and specifically, Saccharomyces cerevisiae, Aspergillus nidulans, Aspergillus fumigatus, Cryptococcus neoformans, Coccidioides immitis, Schizosaccharomyces pombe, Pneumocystis carinii, and Candida albicans. Strains of fungi can be obtained from the ATCC and grown and preserved using methods described in F. Sherman, G. Fink, and J. Hicks, J., Methods in Yeast Genetics, (Cold Spring Harbor Laboratory Press. Cold Spring Harbor, N.Y., 1981).

The RNA fragment employed in the method of the invention to interfere with the expression of the target gene product can be an antisense RNA. The term “antisense RNA” refers to an RNA transcript that is complementary to all or part of a target primary transcript or messenger RNA (mRNA) and that blocks the expression of a target gene. The terms “complementary” and “complementarity” refer to the binding of polynucleotides under permissive salt and temperature conditions by base pairing. Base pairing in a double stranded RNA is a partnership of adenine (A) with uracil (U) and of cytosine (C) with guanine (G) through the formation of hydrogen bonds. The complementarity of an antisense RNA may be with any part of the specific gene transcript; i.e., at the 5′ non-coding sequence, 3′ non-coding sequence, introns, or the coding sequence. The term “RNA transcript” refers to the product resulting from RNA polymerase-catalyzed transcription of a DNA sequence. When the RNA transcript is a complementary copy of the DNA sequence, it is referred to as the primary transcript or it may be a RNA sequence derived from post-transcriptional processing of the primary transcript and is referred to as the mature RNA. “Messenger RNA” refers to the RNA that is without introns and that can be translated into protein by the cell. “Sense” RNA refers to RNA transcript that includes the mRNA and so can be translated into protein by the cell.

Methods for identifying gene targets for asRNA are well known in the art. Suitable methods can be found in or adapted from the teachings of Forsyth et al., Mol. Microbiol. 2002, 43: 1387-1400; DeVito et al., Nat. Biotechnol. 2002, 5: 478-483; and Ji et al., Science 2001, 293: 2266-2269. Suitable asRNA for a target gene can then be constructed by methods well known in the art. For example, asRNA-encoding nucleic acid fragments can be constructed using conventional procedures such as polymerase chain reaction (PCR) amplification, as described in F. M. Ausubel et al., Short protocols in molecular biology, 2^{nd} ed. (John Wiley & Sons, 1992) and in Molecular Cloning: A Laboratory Manual, 3^{rd} edition, edited by J. Sambrook and D. W. Russell (Cold Spring Harbor Laboratory Press, 2000).

The asRNA-encoding nucleic acid can then be incorporated into an appropriate expression vector and the expression vector subsequently transformed or transfected into the host cell. The term “host cell” refers to a prokaryotic or eukaryotic cell which has been transformed or transfected, or is capable of transformation or transfection by an exogenous polynucleotide sequence. Host cells contemplated for use in the practice of the present invention include cells well known in the art. Suitable host cells include yeast cells (e.g., S. cerevisiae, Candida tropicalis, Hansenula polymorpha, Pichia pastoris—see U.S. Pat. Nos. 4,882,279, 4,837,148, 4,929,555 and 4,855,231—and the like), bacterial cells (e.g., Escherichia coli), and the like.

The term “vector” refers to a carrier molecule to which a desired segment of DNA is linked. The vector serves to incorporate foreign DNA into host cells. More particularly, a “vector” is a DNA construct containing a DNA sequence which is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences which control the termination of transcription and translation. The vector may be a plasmid, a phage particle, or simply a potential genomic insert. Once transformed into a suitable host, the vector may replicate and function independently of the host genome, or may in some instances, integrate into the genome itself. The terms “plasmid” and “vector” are sometimes used interchangeably herein, because the plasmid is the most commonly used form of vector at present. However, the invention is intended to include such other forms of vector that serve an equivalent function and are or become known in the art. Typical expression vectors for bacterial cell culture expression, for example, are based on pEPSA5. One type of preferred vector is an episome, i.e., a nucleic acid capable of extra-chromosomal replication.

The term “expression vector” particularly refers to a recombinant nucleic acid molecule that is used to transport heterologous nucleic acid into cells for expression and/or replication thereof. Expression vectors typically come in the form of a plasmid that, upon introduction into an appropriate host cell, results in expression of the inserted DNA. The expression vector may be either circular or linear, and is capable of incorporating a variety of nucleic acid constructs therein. However, the invention is intended to include such other forms of expression vectors capable of equivalent functions and which are or become known in the art.

Expression vectors suitable for use in the practice of the present invention are well known to those of skill in the art and include those that are replicable in eukaryotic cells and/or prokaryotic cells as well as those that remain episomal and those that integrate into the host cell genome. Expression vectors typically further contain other functionally important nucleic acid sequences, such as expression cassettes encoding antibiotic resistance proteins, and the like. Exemplary expression vectors for transformation of E. coli prokaryotic cells include the pET expression vectors (Novagen, Madison, Wis., see U.S. Pat. No. 4,952,496), e.g., pETlla, which contains the T7 promoter, T7 terminator, the inducible E. coli lac operator, and the lac repressor gene; and pET 12a-c, which contains the T7 promoter, T7 terminator, and the E. coli ompT secretion signal. Another such vector is the pIN-IIIompA2 (see Duffaud et al., Meth. in Enzymology 1987, 153: 492-507), which contains the 1pp promoter, the lacUV5 promoter operator, the ompA secretion signal, and the lac repressor gene.

As used herein, the terms “transformation” and “transfection” refer to any of the variety of art-recognized techniques for introducing foreign nucleic acid (e.g., DNA) into a host cell. Suitable means for introducing (transducing) expression vectors containing nucleic acid constructs (e.g., asRNA-encoding nucleic acid) into host cells to produce transduced recombinant cells (i.e., cells containing recombinant heterologous nucleic acid) are well-known in the art. Exemplary techniques are described, for example, in Friedmann,. Science 1989, 244: 1275-1281 and Mulligan, Science 1993, 260: 926-932. Suitable methods for transforming or transfecting host cells can be found in Molecular Cloning: A Laboratory Manual, 3^{rd} edition, edited by J. Sambrook and D. W. Russell (Cold Spring Harbor Laboratory Press, Cold Spring Harbor, N.Y., 2000), and other laboratory manuals. Exemplary methods of transduction include, for example, infection employing viral vectors (see, e.g., U.S. Pat. Nos. 4,405,712 and 4,650,764), calcium phosphate transfection (U.S. Pat. Nos. 4,399,216 and 4,634,665), dextran sulfate transfection, electroporation, lipofection (see, e.g., U.S. Pat. Nos. 4,394,448 and 4,619,794), cytofection, particle bead bombardment, and the like. The heterologous nucleic acid can optionally include sequences which allow for its extrachromosomal (i.e., episomal) maintenance, or the heterologous nucleic acid can be donor nucleic acid that integrates into the genome of the host.

The asRNA-encoding nucleic acid employed in the present invention can be cloned into an expression plasmid. The term “cloning” and variants thereof (e.g., “cloned”) in this context refers to insertion of a nucleic acid fragment into a plasmid vector and the transformation of that resulting cloned plasmid into a laboratory microbial strain such as E. coli. The asRNA-encoding nucleic acid fragments can, for example, be cloned into a plasmid by using standard methods described in F. M. Ausubel et al., Short protocols in molecular biology, 2^{nd} ed. (John Wiley & Sons, 1992). Transformation of the plasmids containing the asRNA-encoding nucleic acid can be accomplished, for example, by calcium phosphate or calcium chloride mediated transfection, electroporation, or infection with a bacteriophage construct. The plasmids can also contain an antibiotic resistance gene (e.g., a chloramphenicol, ampicillin, or carbenicillin resistance gene), which permits the plasmids to be maintained in the cells by continuous selection with the antibiotic. Suitable expression plasmids incorporating asRNA-encoding nucleic acid and an antibiotic resistance gene include pEPSA5. Also suitable is a plasmid that contains a xylose-inducible gene encoding asRNA capable of interfering with the expression of a gene expressing a fatty acid synthase, an aminoacyl-tRNA synthetase, a protein secretase, a peptidyl transferase, a transglycosylase, a transpeptidase, or a ribosomal associated protein.

Bacteria suitable for use in the present invention include bacteria containing an antisense RNA plasmid as described in Forsyth et al., Mol. Microbiol. 2002, 43: 1387-1400, wherein the vector is pEPSA5 containing the pT5X xylose-inducible promoter. The vector is constructed from pC194-derived plasmid pRN5548 (Novick, Methods in Enzymology 1991, 204: 587-636) and a multiple cloning site, rmB T1T2 terminators and the ampicilln resistance gene of the plasmid pLEX5BA (Krause et al, J. Mol. Biol. 1997, 274: 365-380), excluding the ColE1 origin of replication, which is exchanged for a NotI cassette containing the lower copy number p15a origin (Diederich et al., Biotechniques 1994, 16: 916-923). Upstream of the multiple cloning site and terminators is a Gram-positive optimized bacteriophage T5PN25 promoter (LeGrice, Methods Enzynmol. 1990, 185: 201-214) in context with the operator sequence for the Staphylococcus xylosis XylR repressor protein (Schnappinger et al., Microbiol Lett. 1995, 129: 121-127), the gene of which is also included as indicated in the map of pEPSA5.

The method of the present invention can also employ other inducible plasmids, such as the anhydrotetracycline inducible plasmids from Clontech, or arabinose-inducible plasmids from Invitrogen.

The nucleic acid fragment encoding an antisense RNA suitable for use in the present invention can also be incorporated into the genome of the cell using methods known to those skilled in the art. For example, integration of the asRNA-encoding nucleic acid can be accomplished by homologous recombination into the bacterial genome as described in (Nash H. A. in Escherichia coli and Salmonella, Cellular and Molecular Biology, ed. F. C. Neidhardt, (1996) pp 2363-2376). The asRNA-encoding nucleic acid is positioned in the genome with flanking inverted repeat DNA (˜200 bp each), which will allow for efficient excision of the asRNA-encoding nucleic acid under appropriate selection with target-selective compounds or xylose (Craig, N. L. in Escherichia coli and Salmonella, Cellular and Molecular Biology, ed. F. C. Neidhardt, (1996) pp 2339-2362). The stable maintenance of the asRNA-encoding DNA in the bacterial genome will be allowed by the presence of a drug resistance gene (ie, chloramphenicol acetyl transferase) adjacent to the asRNA-encoding nucleic acid. In the absence of chloramphenicol selection and in the presence of a compound inhibiting the asRNA gene product, the genomic copy of the asRNA-encoding nucleic acid will be deleted from the genome, resulting in revertant colony growth.

The following examples serve only to illustrate the invention and its practice. The examples are not to be construed as limitations on the scope or spirit of the invention.

### EXAMPLE 1

### Identification of FabF-Acting Substances in S. Aureus

Part A—Assay Protocol

S. aureus containing S1-1941, a plasmid expressing a xylose-inducible fabF anti-sense RNA (described in WO 00/44906 and in Forsyth et al., Mole. Microbiol. 2002, 43: 1387-1400), are inoculated into an LB broth culture medium supplemented with 34 mg/mL of chloramphenicol, and grown overnight at 37° C. with shaking (225 rpm). The culture is then diluted in the LB medium to a final optical density (λ=600 nm) of 0.3. Diluted cells (750 μL) are then added to a molten LB-agar medium (30 mL) supplemented with xylose (23.6 mM) and glucose (0.19 g/100 mL) and equilibrated at 48° C. The molten mixture is briefly mixed and is then poured onto sterile disposable polystyrene 86×128 mm dishes (NUNC Cat. No. 242811), after which a TSP (=transferable solid phase screening system) casting tray (NUNC Cat. No. 445497) is placed on top to cast wells. The dishes are allowed to cool down to room temperature for about 15 minutes and are then stored for no more than 2 days at 4° C. until ready for sample dispensing. Samples (10 μL) of one or more test substances (500 μM) and samples of controls, each dissolved in 100% DMSO, are subsequently dispensed into wells and the plates incubated overnight at 37° C. The incubated plates are then inspected visually for the appearance of revertant colonies to determine whether the test substance(s) act at the fabF protein.

One control is an antibiotic that targets the fabF gene, and another control is an antibiotic that does not target the fabF gene. The fabF-targeting control will produce revertant colonies in and around a zone of inhibition, and the non-fabF targeting control will produce a clear zone of inhibition (i.e., devoid of revertant colonies). The zones of inhibition of the test substances are compared to those of the controls to screen for fabF-acting substances. In the event the plate does not display the appropriate inhibition zones for the controls (e.g., the plate displays promiscuous colonies over the entire assay plate due to too high a dose of xylose and/or too long an incubation time), the plate is not valid for screening and is discarded.

Part B—Assay Results

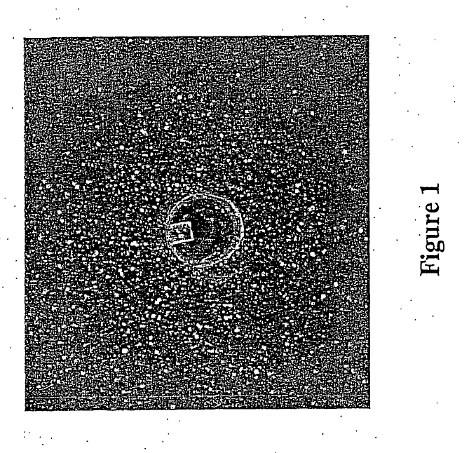

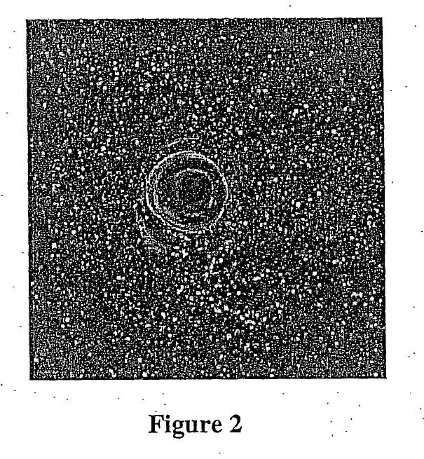

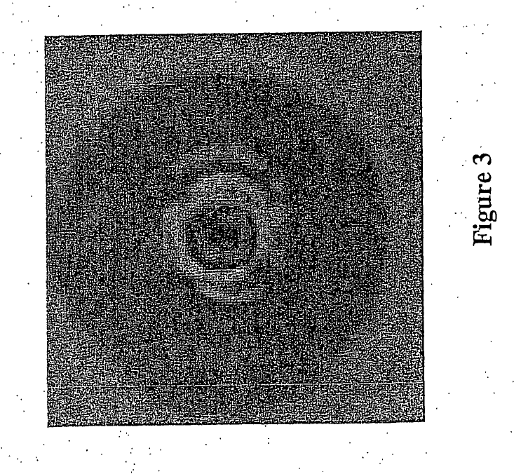

The assay protocol set forth in Part A was employed to test whether thiotetramycin is a fabF-acting antibacterial agent. A 10 μL sample of thiotetramycin dissolved in 100% DMSO (1 mg/mL) was dispensed into one well of the plate. In addition, a 10 μL sample of cerulenin in 100% DMSO (20 μg/mL) and a 10 μL sample of kanamycin in water (100 μg/mL) were respectively dispensed into two wells as controls. After incubation as described in Part A, the plates were inspected visually for zones of bacterial growth. Thiotetramycin resulted in the growth of revertant colonies which appear as small white foci of growth in and around a zone of growth inhibition, as shown in FIG. 1. Cerulenin, an antibiotic that targets the fabF gene, also resulted in the growth of revertant colonies in and around a zone of growth inhibition, as shown in FIG. 2. On the other hand, kanamycin, an antibiotic that does not target the fabF gene, resulted in a clear zone of no growth and no appearance of revertant colonies, as shown in FIG. 3. The results show that the test substance, thiotetramycin, is an antibacterial agent that that selectively targets the fabF protein.

### EXAMPLE 2

### Identification FabF-Acting Substances in S. Aureus from Natural Products

Substance-producing microorganisms were prepared as follows: Soil samples containing unidentified microorganisms were suspended in sterile water, and the suspension was applied to the surface of a culture dish containingISP-agar medium (Difco/Becton Dickinson Inc.). After surface-drying, the dish was incubated at 27° C. for 2-7 days until visible growth was obtained. Strips of agar containing microorganism were cut and removed from the dish for application onto assay plates prepared as described below. Alternatively, organic solvent (acetone) extracts of these microorganism were prepared and the solvent evaporated from the crude extract and resuspended in 100% DMSO.

S. aureus cells containing S1-1941 were cultured and assay plates were prepared as described in Example 1, except that no wells were cast in the agar. Following solidification of the S. aureus-containing agar plates, fresh agar strips containing the growing microorganisms were spotted on the surface of the assay plates, after which the plates were incubated for 24 hours at 30° C. In the case of testing a crude extract, the assay procedure is as described in Example 1 (i.e. samples were dispensed in precast wells).

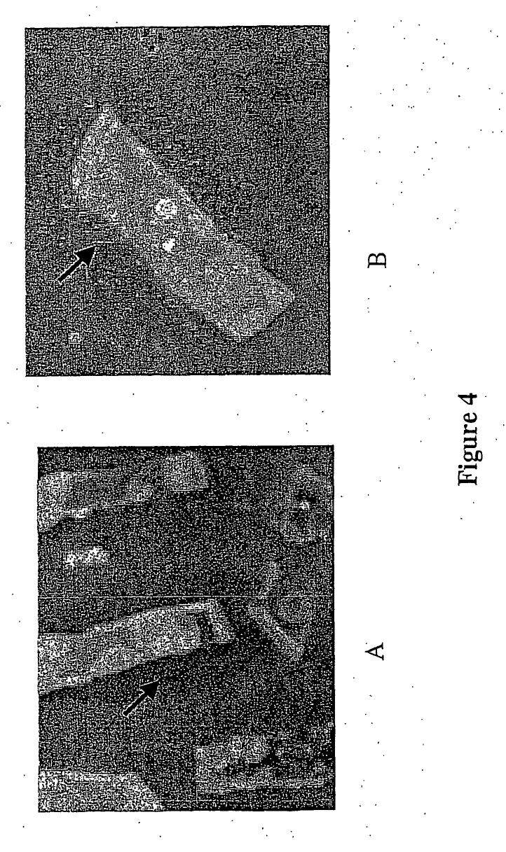

FIG. 4 shows the post-incubation surfaces of representative assay plates. FIG. 4A shows several strips, wherein the central strip has a clear zone of no growth, as indicated by the arrow. This indicates that at least one of the microorganisms in the central strip produces an antibacterial product that does not target fabF. FIG. 4B is a close up view of a single strip, which is encompassed by a zone of no growth (indicated by the arrow), except for revertant colonies appearing as small white foci of growth in and/or around the zone. Accordingly, at least one of the microorganisms in the strip in 4B produces a fabF-specific antibacterial agent.

FIG. 5 shows the growth inhibition zone around a precast well containing the undefined crude aforementioned extract for identifying fabF-acting substances in S. aureus. The zone has several visually observable revertant bacterial colonies surrounding a central zone of no visually observable bacterial growth. In this case, the clear zone contains a growth inhibitor that does not selectively inhibit fabF and the revertant colony-containing peripheral zone contains a growth inhibitor that selectively inhibits fabF.

### EXAMPLE 3

### Identification FabF-Acting Substances in S. Aureus in a Liquid Format Using the Green Fluorescent Protein

The green fluorescent protein (GFP) reporter gene was used to identify fabF antisense revertants in liquid culture by the following method: S. aureus containing a plasmid encoding the antisense fabF DNA was used to develop an inducible GFP reporter system, where the GFP gene is transcriptionally silent until the fabF antisense containing plasmid DNA is deleted or rearranged. A plasmid, pM310, was constructed that consists of the fabF antisense vector (Forsyth et al, 2002), with a tetracycline repressor gene and its promoter (Geissendorfer et al., Appl. Microbiol. Biotechnol 1990, 33: 657-663) inserted downstream of the fabP antisense DNA. A second plasmid, pM302, was constructed that consists of the E. coli vector pUC19 (Gibco BRL) joined to the S. aureus vector pUB110 (ATCC 37015), through the EcoR1 and AatII restriction sites. At the pUC19 multicloning site, a GFP gene was inserted into the BamH1 and SalI restriction sites and a tetracycline operator containing promoter (Geissendorfer et al, 1990) was cloned into the EcoR1 and BamH1 sites. A S. aureus strain, SafabF/GFP, was constructed that contained both plasmids pM310 and pM302 and was resistant to chloramphenicol and kanamycin. SafabF/GFP was sensitive to xylose induction resulting in an increased sensitivity to cerulenin. In the presence of cerulenin, resistant revertants will be selected for, which have deleted the antisense vector, and along with this deletion, lost the tetracycline repressor gene. Since the tetracycline repressor suppresses the GFP transcription, deletion of the tetracycline repressor results in the transcription of the GFP and the production of green cells. Revertants can be measured as a function of fluorescent readout relative to cell number.

To quantitatively measure the fabF revertants, a liquid assay was developed, where various concentrations of xylose were tested with various concentrations of the following test compounds: cerulenin, thiostrepton and ampicillin. A 20 hour culture of SafabF/GFP was grown in LB (Becton Dickinson, #244620), kanamycin (100 μg/mL) and chloramphenicol (34 μg/mL), and adjusted to 0.03 OD A600 in a 1 cm cuvette. A 96-well plate was used to measure growth of the culture and final absorbance after 16 hours of incubation at 37° C. In each well containing 100 μl 2× mix (200 μg/ml Kanamycin, 0.4% glucose, 0.4% DMSO, 2× mM xylose and 2× concentration of test compounds), 100 μl of the adjusted culture were added to form the final 200 μl volume. The final absorbance as read at 590 nm (A590) and fluorescence of the GFP was measured by excitation at 485 nm wavelength and emission wavelength at 535 nm (E535), using a Tecan Spectrafluor Plus.

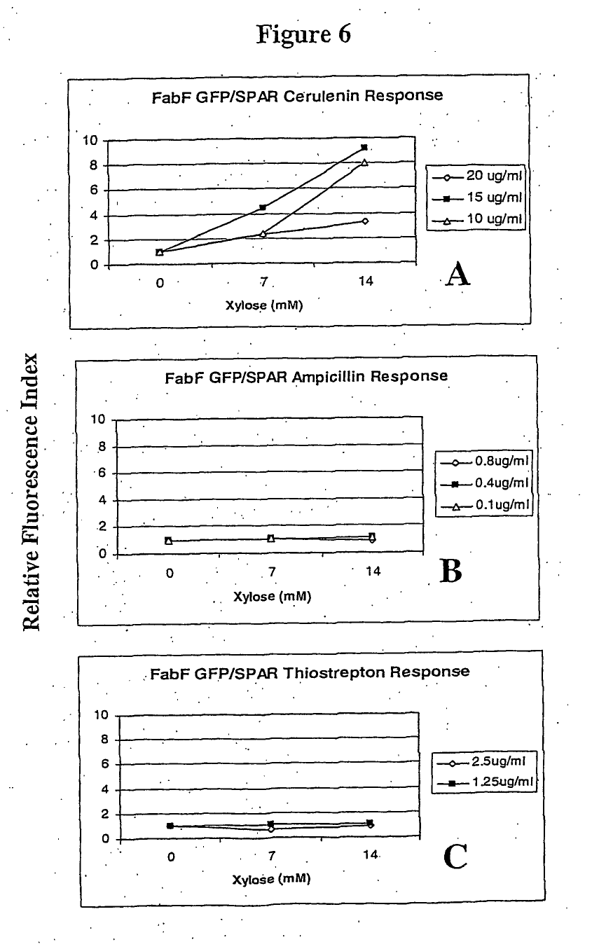

To detect a specific GFP response in the presence of a fabF selective inhibitor, cerulenin, the Relative Fluorescence Index (RFI) was calculated for each drug treatment and for each xylose treatment. The RFI is defined as E535/A590 ratio, normalized to the corresponding ratio calculated on no-xylose control cells within each drug test group. For instance, with 15 μg/ml cerulenin treatment, the E535/A590 ratio was calculated for 0, 7 and 14 mM xylose treated cells, and the ratio for each were divided by the E535/A590 ratio from the no-xylose treated cells. The results are shown in FIG. 6A, where the SafabF/GFP cells were treated with the fabF inhibitor cerulenin. Analogous results for treatment of the cells with two nonspecific antibiotics, ampicillin and thiostrepton, are shown in FIGS. 6B and 6C respectively. Drug concentrations were chosen that approximated the 50% inhibition of growth during the 16 hour incubation period, in the no-xylose treated cells. Clearly, for each concentration of cerulenin the RFI increased with increasing xylose concentration with a maximum change of 9-fold observed at 14 mM xylose and 15 μg/ml cerulenin. No increase in RFI was observed for the non-fabF antibiotics, thiostrepton and ampicillin. The selective increase in the RFI for cerulenin treated cells indicates that the GFP readout can be used to detect selective fabF drugs in liquid culture.

While the foregoing specification teaches the principles of the present invention, with examples provided for the purpose of illustration, the practice of the invention encompasses all of the usual variations, adaptations and/or modifications that come within the scope of the following claims.

References are made throughout this application to various published documents in order to more fully describe the state of the art to which this invention pertains. The disclosures of these references are herein incorporated by reference in their entireties.

What is claimed is:

1. A method for testing whether a substance is or contains a cell growth inhibitor that acts by selectively inhibiting the function of a gene product required for cellular growth or survival, wherein the method comprises:
(A) providing recombinant cells that are capable of expressing an RNA fragment that interferes with the expression of the gene product;
(B) growing the recombinant cells in a nutrient medium in the presence of the test substance and under conditions which result in (i) the expression of the RNA fragment and down regulation of the synthesis of the gene product and (ii) the loss by the cells of the capability to express the RNA fragment; and
(C) analyzing the resulting cell growth; wherein:
(1) if there is essentially no cell growth due to the death of all or substantially all of the cells, then the test substance is or contains a growth inhibitor that does not selectively inhibit the targeted gene product;
(2) if there is essentially no inhibition of cell growth due to the survival and growth of all or substantially all of the cells, then the test substance is not or does not contain a growth inhibitor; or
(3) if there is growth inhibition due to the death of a substantial portion of the cells accompanied by the survival and growth of revertant cells having no capability to express the RNA fragment, then the test substance is or contains a growth inhibitor that selectively inhibits the targeted gene product.

2. The method according to claim 1, wherein the RNA fragment expressed by the cells in Step A comprises an antisense RNA.

3. The method according to claim 2, wherein the antisense RNA is encoded in a plasmid contained in the recombinant cells.

4. The method according to claim 2, wherein the antisense RNA is encoded in DNA in the genome of the recombinant cells.

5. The method according to claim 1, wherein the recombinant cells are selected from the group consisting of bacterial strains and fungal strains.

6. The method according to claim 5, wherein the cells are a strain of bacteria and the substance is being tested to determine whether it is or contains an antibacterial agent that acts by selectively inhibiting the function of a gene product required for the growth or survival of the bacteria.

7. The method according to claim 6, wherein the RNA fragment capable of being expressed by the bacteria in Step A comprises an antisense RNA encoded in a plasmid contained in the bacteria.

8. The method according to claim 1, wherein the nutrient medium in which the recombinant cells are grown in Step B is a semi-solid medium inoculated with the test substance; and analyzing the resulting cell growth in Step C; wherein:
(1) if the semi-solid medium exhibits a clear zone indicative of essentially no growth due to the death of all or substantially all of the cells, then the test substance is or contains a growth inhibitor that does not selectively inhibit the targeted gene product;
(2) if the semi-solid medium does not exhibit a zone of no growth, due to the survival and growth of all or substantially all of the cells, then the test substance is not or does not contain a growth inhibitor; or
(3) if the semi-solid medium exhibits a zone of no growth except for one or more small cell colonies in the zone due to the survival and growth of revertant cells having no capability to express the RNA fragment, then the test substance is or contains a growth inhibitor that selectively inhibits the targeted gene product.

9. The method according to claim 8, wherein the recombinant cells are a strain of bacteria and the test substance is being analyzed to determine whether it is or contains an antibacterial agent that acts by selectively inhibiting the function of a gene product required for the growth or survival of the bacteria.

10. The method according to claim 8, wherein the semi-solid medium is an agar plate or an agarose plate.

11. The method according to claim 1, wherein:
the RNA fragment expressed by the cells in Step A comprises an antisense RNA encoded in a first plasmid, said first plasmid also encoding a repressor gene that regulates a reporter gene integrated into the genome of the cells or contained in a second plasmid;
the nutrient medium in Step B is a liquid medium;
the loss by the cells of the capability to express the RNA fragment in Step B is accompanied by the loss of the capability of the repressor to regulate the reporter gene; and
in the analysis of cell growth in Step C:
(1) if there is essentially no cell growth due to the death of all or substantially all of the cells, then the test substance is or contains a growth inhibitor that does not selectively inhibit the targeted gene product;
(2) if there is essentially no inhibition of cell growth due to the survival and growth of all or substantially all of the cells, said cells having no expression of the reporter gene, then the test substance is not or does not contain a growth inhibitor; or
(3) if there is growth inhibition due to the death of a substantial portion of the cells in the liquid medium accompanied by the survival and growth of revertant cells having no capability to express the RNA fragment but having expression of the reporter gene, then the test substance is or contains a growth inhibitor that selectively inhibits the targeted gene product.

12. The method according to claim 11, wherein the reporter gene is encoded in a second plasmid and encodes a fluorescent protein; and the survival and growth of revertant cells is indicated by the detection of fluorescence from the fluorescent protein.

13. The method according to claim 12, wherein the cells are a strain of bacteria; and the antisense RNA is a xylose-inducible antisense RNA encoded in the first plasmid and is capable of interfering with the expression of a gene selected from the group consisting of fabF, pheT, murA, secA, dnaC, ileS, ftsZ, secI, polC, dnaE, gyrE, gyrA, murB, rpl, parE, and parC.

14. The method according to claim 12, wherein the analysis of cell growth in Step C comprises measuring the level of fluorescence relative to the number of cells, and the number of cells is determined by measuring absorbance.

15. A method for testing whether a substance is or contains an antibacterial agent that acts by selectively inhibiting the function of a gene product required for the growth or survival of a strain of bacteria, wherein the method comprises:
(A) providing recombinant cells of the bacterial strain that are capable of expressing an RNA fragment that interferes with the expression of the gene product;
(B) growing the recombinant cells in a nutrient medium in the presence of the test substance and under conditions which result in (i) the expression of the RNA fragment and the down regulation of synthesis of the gene product and (ii) the loss by the cells of the capability to express the RNA fragment; and
(C) analyzing the resulting cell growth; wherein:
(1) if there is essentially no cell growth due to the death of all or substantially all of the cells, then the test substance is or contains an antibacterial agent that does not selectively inhibit the targeted gene product;
(2) if there is essentially no inhibition of cell growth due to the survival and growth of all or substantially all of the cells, then the test substance is not or does not contain an antibacterial agent; or
(3) if there is growth inhibition due to the death of a substantial portion of the cells accompanied by the survival and growth of revertant bacterial cells having no capability to express the RNA fragment, then the test substance is or contains an antibacterial agent that selectively inhibits the targeted gene product.

16. The method according to claim 15, wherein the RNA fragment expressed by the cells in Step A comprises an antisense RNA.

17. The method according to claim 16, wherein the antisense RNA is encoded in a plasmid contained in the cells.

18. The method according to claim 16, wherein the antisense RNA is encoded in DNA in the genome of the cells.

19. The method according to claim 15, wherein the bacterial strain is selected from the group consisting of Staphylococcus, Streptococcus, Enterococcus, Escherichia, Klebsiella, Haemophilus, Enterobacter, Acinetobacter, Bacillus, Stenotrophomonas, Burkholderia, Salmonella, and Pseudomonas.

20. The method according to claim 19, wherein the bacterial strain is selected from the group consisting of Streptococcus pneumonia, Staphylococcus aureus, Enterococcus faecalis, Enterococcus faecium, Escherichia coli, Klebsiella pneumoniae, Haemophilus influenzae, Enterobacter cloacae, Pseudomonas aeruginosa, Acinetobacter baumanii, Bacillus subtilis, Stenotrophomonas maltophilia, Salmonella typhimurium, and Burkholderia cepacia.

21. The method according to claim 20, wherein the bacterial strain is a Staphylococcus aureus containing a plasmid expressing a xylose-inducible anti-sense RNA for a gene required for the survival or growth of Staphylococcus aureus.

22. The method according to claim 21, wherein the xylose-inducible anti-sense RNA expressed by the plasmid is for a gene selected from the group consisting of fabF, pheT, murA, secA, dnaC, ileS, ftsZ, secI, polC, dnaE, gyrE, gyrA, murB, rpl, parE, and parC.

23. The method according to claim 15, wherein the target gene of the RNA fragment is encoded to express a fatty acid synthase, an aminoacyl-tRNA synthetase, a protein secretase, a peptidyl transferase, a transglycosylase, a transpeptidase, or a ribosomal associated protein.

24. The method according to claim 15, wherein the nutrient medium in which the recombinant cells are grown in Step B is a semi-solid medium inoculated with the test substance; and analyzing the resulting cell growth in Step C; wherein:
(1) if the semi-solid medium exhibits a clear zone indicative of essentially no growth due to the death of all or substantially all of the cells, then the test substance is or contains an antibacterial agent that does not selectively inhibit the targeted gene product;
(2) if the semi-solid medium does not exhibit a zone of no growth, due to the survival and growth of all or substantially all of the cells, then the test substance is not or does not contain an antibacterial agent; or
(3) if the semi-solid medium exhibits a zone of no growth except for one or more small cell colonies in the zone due to the survival and growth of revertant cells having no capability to express the RNA fragment, then the test substance is or contains an antibacterial agent that selectively inhibits the targeted gene product.

25. The method according to claim 24, wherein the semi-solid medium is an agar plate or an agarose plate.

26. A method for testing whether a substance is or contains an antibacterial agent that acts by selectively inhibiting the function of a gene product required for the growth or survival of a strain of bacteria, wherein the method comprises:
(A) providing recombinant cells of the bacterial strain containing a plasmid that is capable of expressing an antisense RNA that interferes with the expression of the gene product; and
(B) growing the recombinant cells on an agar plate in the presence of the test substance and under conditions which result in (i) the expression of the antisense RNA and the down regulation of synthesis of the gene product and (ii) the loss by the cells of the capability to express the antisense RNA; and
(C) analyzing the resulting cell growth; wherein:
(1) if the agar plate exhibits a clear zone indicative of essentially no growth due to the death of all or substantially all of the cells, then the test substance is or contains an antibacterial agent that does not selectively inhibit the targeted gene product;
(2) if the agar plate does not exhibit a zone of no growth, due to the survival and growth of all or substantially all of the cells, then the test substance is not or does not contain an antibacterial agent; or
(3) if the agar plate exhibits a zone of no growth except for one or more small cell colonies in the zone due to the survival and growth of revertant cells having no capability to express the RNA fragment, then the test substance is or contains an antibacterial agent that selectively inhibits the targeted gene product.

27. The method according to claim 26, wherein the plasmid is a plasmid that is induced to express the antisense RNA and that is encoded for resistance to an antibiotic.

28. The method according to claim 27, wherein in Step B (i) the expression of the antisense RNA and the down regulation of the gene product is obtained by growing the cells in the presence of an effective amount of an antisense RNA-inducer and (ii) the loss by the cells of the capability to express the antisense RNA is obtained by growing the cells in the absence of the antibiotic to which the plasmid is resistant.

29. The method according to claim 28, wherein the antisense RNA is a xylose-inducible antisense RNA capable of interfering with the expression of a gene expressing a fatty acid synthase, an aminoacyl-tRNA synthetase, a protein secretase, a peptidyl transferase, a transglycosylase, a transpeptidase, or a ribosomal associated protein.

30. The method according to claim 29, wherein the antisense RNA is a xylose-inducible antisense RNA capable of interfering with the expression of a gene selected from the group consisting of fabF, pheT, murA, secA, dnaC, ileS, ftsZ, secI, polC, dnaE, gyrE, gyrA, murB, rpl, parE, and parC.

31. The method according to claim 30, wherein the bacteria is Staphylococcus aureus.
